(19) United States
(12) Patent Application Publication
(10) Pub. No.: US 2015/0309193 A1
(43) Pub. Date: Oct. 29, 2015

(54) Radiation Beam Direction Determination Using Three-Dimensional Measurement Device

(71) Applicant: SUN NUCLEAR, Melbourne, FL (US)

(72) Inventors: Jakub Kozelka, Melbourne, FL (US); William E. Simon, Melbourne, FL (US)

(21) Appl. No.: 14/559,797

(22) Filed: Dec. 3, 2014

Related U.S. Application Data
(63) Continuation of application No. 14/044,726, filed on Oct. 2, 2013, now abandoned.
(60) Provisional application No. 61/708,906, filed on Oct. 2, 2012.

(51) Int. Cl. G01T 1/29; A61N 5/10

(52) U.S. Cl. CPC G01T 1/2907; A61N 5/1075

(57) ABSTRACT

A method has been developed to reconstruct angle of the radiation field using a 3D measurement device. The 3D measurement device is positioned in the radiation beam. The novel method uses measured values and information about attenuation in the 3D detector and calculates direction of the primary beam.

### CROSS-REFERENCE TO RELATED APPLICATION

This application claims the benefit of U.S. Provisional Application Ser. No. 61/708,906, filed on Oct. 2, 2012, the contents of which are herein incorporated by reference in their entirety.

### FIELD OF THE INVENTION

The present invention relates to medical diagnosis and treatment using ionizing radiation, and more particularly, to systems and methods for ensuring the proper delivery of such radiation.

### BACKGROUND OF THE INVENTION

Radiation detector arrays are used to measure the radiation intensity distribution exiting the beam port of a linear accelerator. Such beams generally consist of x-rays or electrons and are used, among other applications, to treat cancer. Ionizing radiation ionizes matter along its path. Direct interaction of ionizing radiation with DNA and also interaction of ionized water electrochemical products with DNA may lead to un-repairable damage of DNA. Un-repairable DNA damage leads to cell death. The probability of heavy damage to DNA per delivered energy is not large and therefore the amount of dose (energy per mass) has to relatively high, while targeted to minimize dose to healthy tissue.

During treatment, the beam port is adjusted to shape the beam to the tumor shape. To minimize the dose to healthy tissue, the dose is delivered using more fields, or even by rotating the beam port.

The linear accelerator is a machine that can accelerate electrons up to 18 MeV or more. These accelerated electrons can be used for creating x-ray radiation by putting a piece of dense matter in the electron's path. Electrons emit photons when decelerate. The x-ray beam is primarily shaped using two pairs of jaws, which can include pieces of tungsten up to 10 cm wide. The beam is further collimated by a multi-leaf collimator, composed of tungsten slabs a few millimeters thick and up to 10 cm wide. The beam intensity depends on the collimation system, selected beam energy and dose rate, which—in the case of electrons—is equivalent to the accelerated electron's current.

Manufacturers of such accelerators include Varian Corporation (Palo Alto, Calif., USA), Elekta AB (Stockholm, Sweden), Siemens (Munich, Germany).

The linear accelerator rotates on a gantry for precise delivery to the patient lying on a table. Therefore, precise data on the beam shape, beam direction and intensity is important for proper dose control to the tumor. Various types of 2D detectors were developed to measure beam shape and intensity of the modulated beam. None of these detectors is capable of measuring beam direction.

An early true three-dimension (3D) detector used in radiation therapy was BANG gel, but this type of detector is not widely used for other problems. The first electronic device claimed to be able to measure 3D dose was Delta4 produced by ScandiDos AB (Uppsala, Sweden). Delta 4 is able to measure beam shape and beam intensity, but it is not able to measure beam direction. The information about beam direction is provided by an independent inclinometer attached to the gantry structure. However, there is ambiguity between beam direction and position of the gantry, which is not necessarily constant at all angles.

Another 3D device that is capable of measuring 3D dose under certain circumstances is ArcCHECK from Sun Nuclear Corp. (Melbourne, Fla., USA). This detector array has 1386 detectors placed in a cylindrical phantom. The diodes are placed on a spiral. Diodes are separated by 1 cm and the spiral pace is 1 cm per revolution. The phantom shape and placement of the detectors make the unit coherent to the beam; i.e., the detector array is invariant to the gantry rotation.

### SUMMARY OF THE INVENTION

The objective of the present invention is to provide a method for determining information about beam direction using a 3D array that measures radiation beams such as but not limited to photon beams, x-ray and electron beams.

A 3D detector measures dose in 3D volume, or part of the 3D volume. Even 4D detectors can be turned into 3D detectors by analyzing every time segment. When the detector is being irradiated, most of the primary photons will keep their direction, although some partial scattering will occur. A 3D detector exposed to radiation beam will detect photons entering the detector and photons exiting the detector. Along the beam's path, the beam is attenuated, so the 3D detector will measure different intensity at the entry side and at the exit side of the 3D detector with respect to the beam direction.

To understand this, it is helpful to visualize a virtual, projection plane inside the detector that cuts the detector in half. There is not such a physical plane, but it the virtual plane is a mathematical tool. It is advantageous to put the projection plane at such a point that will cut the 3D detector into two identical pieces, but it is not necessarily required. For a cylindrical array, that virtual plane would be the center of the cylinder. The reason is to keep the same amount of detectors “above” the projection plane and “under” the projection plane.

The projection plane is used for creating two images. One image is created by projecting the “above” detector volume to the projection plane and the second image is created by projecting the “under” detector volume on the projection plane.

The projected images are based on the known orientation of the detector in the space, the known orientation of the projection plane and an estimated position of the source of radiation. It is advantageous to initially orient the projection plane to be normal to the estimated position of the beam source, but not necessarily required. The projected images are generated by projecting the halves of the detector volume to the plane using analytic geometry math. The projection has to be further adjusted for beam attenuation.

The beam attenuation is dependent on the 3D detector shape and material. The beam attenuation within the 3D detector can be calculated by solving beam transport equations or using other techniques, but the easiest way is to measure it. The FIG. 3 demonstrates measured beam attenuation in the ArcCHECK detector array.

Two images are created by projecting two halves of the 3D detector volume on the projection plane and adjusting them for beam attenuation.

If the estimated position of the source was in coincidence with the real beam source, then these two images are going to be identical (FIG. 4). If the estimated position is different then the real beam position, then these two images are going to be different (FIG. 5).

The two images are never perfectly identical, because the detector array is affected by white noise, uncertainty in calibration factors, slight misalignment, and other parameters, that contribute to the overall uncertainty of the measurement. It is useful to calculate a difference between the images.

$$Diff ( α ) = {\sum }_{i} {\sum }_{j} {( {image1}_{i , j} - {image2}_{i , j} )}^{2} , \qquad Equation 1$$

where α is angle (or position) of the estimated beam source.

The difference between two images at a particular angle of the projection plane shows a level of identity of two pictures. In ideal case, two identical images have Diff=0. The Diff function (FIG. 6) has several minimums and one global minimum. The global minimum represents a point, when the estimated beam source position corresponds with the real source position.

As presented in the FIG. 6, different energies of the beam have different attenuation profiles. It is not necessary to know initially what the energy of the beam is. It is possible to calculate the Diff functions for all energies and select the energy at which the Diff's global minimum is the lowest from all global minimums. The result of the method is then information about the beam source position and energy of the beam.

The ArcCHECK phantom can be used as a hollow cylinder or a full cylinder. Calculating the diff functions for these two scenarios and all beam energies, it is possible to get information about the type of phantom, beam energy and beam source position, just by selecting the lowest global minimum from all diff functions.

### BRIEF DESCRIPTION OF THE DRAWINGS

FIG. 1: LINAC coordination system;

FIG. 2: ArcCHECK coordination system;

FIG. 3: Beam attenuation in ArcCHECK. 6 MV beam attenuation in ArcCHECK on CAX location (Y=0) with respect to the beam located at 0 degrees. The ArcCHECK coordinate system corresponds to the Linac coordinate system.

FIG. 4 illustrates a projection plane with estimated source position coincident with the real source position;

FIG. 5 illustrates a projection plane with estimated source position not coincident with the real source position;

FIG. 6 charts the Diff function for two different ArcCHECK conditions with and without a plug;

FIG. 7 graphically illustrates calculation of the projection plane transformation Matrix™; and

FIG. 8 is a graphical representation of the projection plane transformation Matrix™.

### DETAILED DESCRIPTION OF PREFERRED EMBODIMENTS

Before explaining the disclosed embodiment of the present invention in detail it is understood that the invention is not limited in its application to the details of the particular arrangement shown since the invention is capable of other embodiments. Also, the terminology used herein is for the purpose of description and not of limitation.

The details are explained on the ArcCHECK detector array, but the same approach is applicable to any 3D detector.

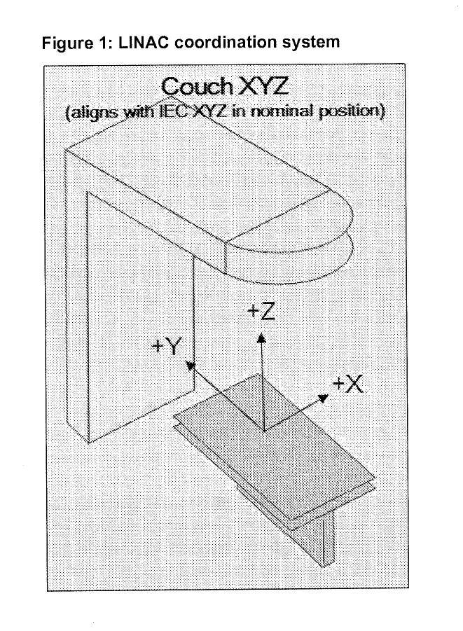

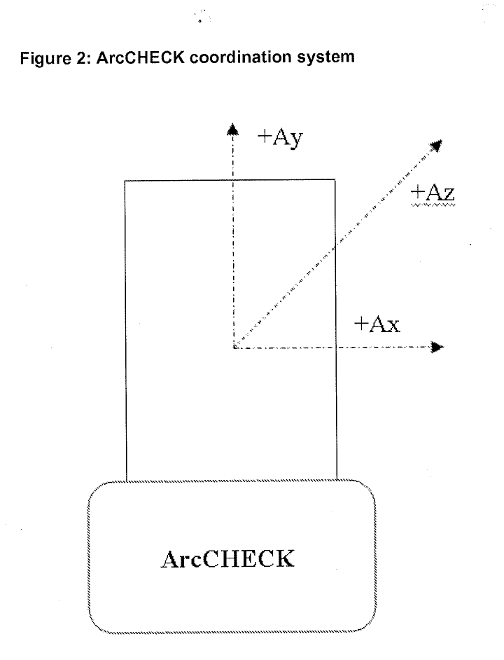

All the equations are in the ArcCHECK coordination system (FIG. 2) and LINAC coordination system (FIG. 1).

For an initial example, it is assumed that ArcCHECK coordinates are perfectly aligned with the Linac coordinates, i.e. [Ax;Ay;Az]=[x;y;z]=[0;0;0] and axis alignment: Ax=x, Ay=y (no rotation is applied).

The ArcCHECK detector array can be described as a cylinder.

Ax²+Az²=radius², Equation 2

where radius of ArcCHECK is 10.4 cm.

The gantry rotates in the XZ plane at a distance from the center (SAD). It can be expressed as the Equation 2, but it is more beneficial to express it in the cylindrical coordinate system:

x=SAD·sin(ω)

z=SAD·cos(ω) Equation 3

SAD is a fixed value and the only variable is then angle w that defines position of the radiation beam.

Defining the projection plane as a plane that is parallel to the AY axis (as well as Y axis in this case), goes through the point [Ax;Ay;Az]=[x;y;z]=[0;0;0] and is always normal to the angle ω. The construction of the plane under these conditions assures that the ArcCHECK detector array is cut in half.

The next step is to calculate images of the halves of ArcCHECK. The method described here is based on creation of projection plane transformation matrixes and using this transformation matrix for generating the images. This method has the advantage of being efficient from a computing perspective, although other methods are possible.

Measured Value Function

The ArcCHECK detector array is a cylinder and can be described very efficiently using cylindrical coordinate system. We can define a function MV (measured value) that returns measured value at given cylindrical coordinates:

Measured value=MV(Y,γ), Equation 4

where Y is distance on Y axis and γ is angle.

If the density of detectors is not infinite, one can select the closest detector, or use several types of interpolations.

Projection Plane Transformation Matrix (TM)

The projection plane transformation matrix is a 3D matrix with dimensions (K, L, 3), where K and L represent size of the images on the projection plane the plane being measured in pixels. The third dimension contains three parameters: Shift of the point on the Y axis, angle α and an attenuation parameter (AT).

The plane transformation matrix has to be calculated only once and it has to be calculated for both halves of ArcCHECK. The transformation matrix can be calculated for one, fixed position ignoring the other angles, because the system of gantry and ArcCHECK array is invariant to rotation around Y axis (or Ay axis).

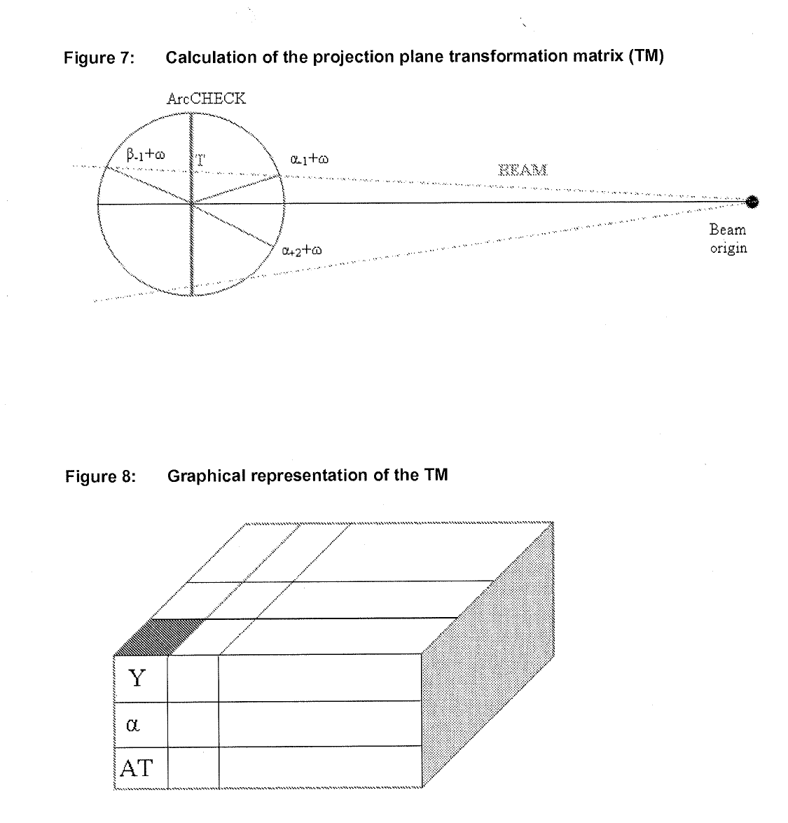

FIG. 7 demonstrates one, fixed position of the system Linac and ArcCHECK. The FIG. 7 also demonstrates why it is necessary to calculate the transformation matrix only once and why it can be calculated at one, fixed angle.

The top beamlet crosses ArcCHECK at α₋₁+ω and β₋₁+ω, where these angles are angles in the ArcCHECK coordinate system. The top beamlet crosses the projection plane in the point T. The point T has always the same location on the projection plane at all gantry angles ω, because the projection plane is always normal to the beam. At all gantry angles ω, the beamlet crosses the ArcCHECK array at angle α₋₁ with respect to the projection plane. Target coordinates are: Tartget=T[x₂;y₂;z₂]=T[SAD;0;0]]

Each beamlet can be described as a line:

x=(x₂−x₁)·t+x₁, y=(y₂−y₁)·t+y₁, z=(z₂−z₁)·t+z₁. Equation 5

Solving for intersections between the beamlet and ArcCHECK detector array (Equation 2 and Equation 3) and taking into account that coordination systems of ArcCHECK and LINAC are identical, we can put Ax=x, Ay=y, Az=z.

((x₂−x₁)·t+x₁)²+((z₂−z₁)·t+z₁)²=radius² Equation 6

Solving for t:

t²└(x₂−x₁)²+(z₂−z₁)²┘+t·2·[x₁·(x₂−x₁)+z₁·(z₂−z₁)]+x₁²+z₁²−radius²=0 Equation 7

Equation 7 is simplified by using the coordinates of the target (beam source). It is further simplified by taking into account that the projection plane x coordinate is 0.

t²·[SAD²+z₁²]+t·2·[−z₁²]+z₁²−radius²=0 Equation 8

Note: Equation 8 has only one variable, which is z₁. This variable is used for filling the image with values. The variable z₁ corresponds to the image size K—(the image has K×L number of pixels, as was defined earlier). The K variable corresponds to Y axis and L variable corresponds to Z axis in the Linac coordination system.

Equation 8 gives two solutions for each beamlet going through point z₁. Using results of the Equation 8 and Equation 2, one can calculate cross sections of the beamlet and ArcCHECK detector array. The result is in Cartesian coordinates, and should be converted into cylindrical coordinates.

$$α = arc tan ( \frac{z}{x} ) = arc tan ( \frac{- {z}_{1} \cdot {t}_{1} + {z}_{1}}{SAD \cdot {t}_{1}} ) \\ β = arc tan ( \frac{z}{x} ) = arc tan ( \frac{- {z}_{1} \cdot {t}_{2} + {z}_{1}}{SAD \cdot {t}_{2}} ) \\ {Y}_{1} = - {y}_{1} \cdot {t}_{1} + {y}_{1} , and {Y}_{2} = - {y}_{1} \cdot {t}_{2} + {y}_{1} \qquad Equation 9$$

For each pixel location, with coordinates y₁ and z₁, one can calculate angles α and β and also coordinate Y on the ArcCHECK detector array using Equation 9. These values are stored in the two projection plane transformation matrixes. One matrix contains Y₁ and α₁, the second contains Y₂ and α₂.

Graphical representation of the TM matrix is shown on the FIG. 8. The red spot represents one pixel of one of the images.

Attenuation Factor

The third value (attenuation parameter—AT) can be calculated in theory using some beam transport calculation or other techniques, but it is easier to measure it. The measurement is preferably done for all energies and in case of ArcCHECK, for scenarios with and without a plug.

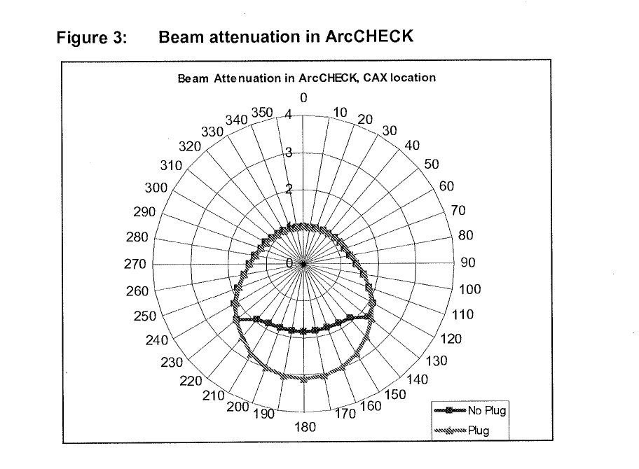

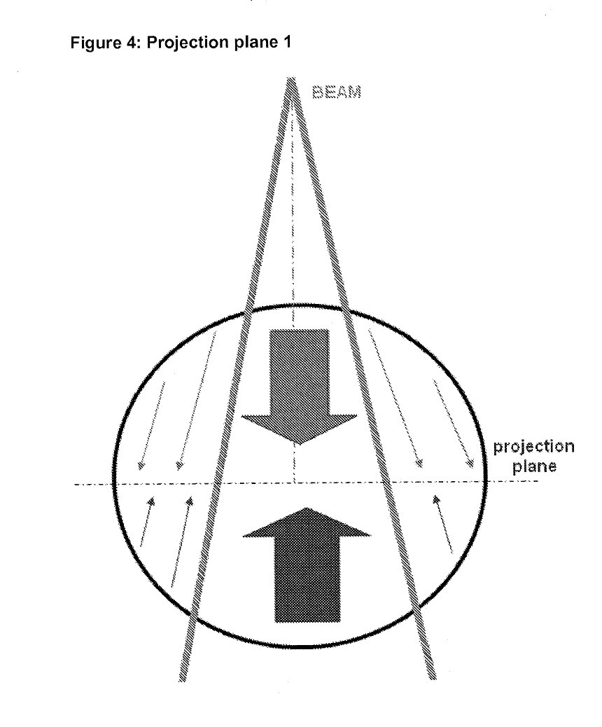

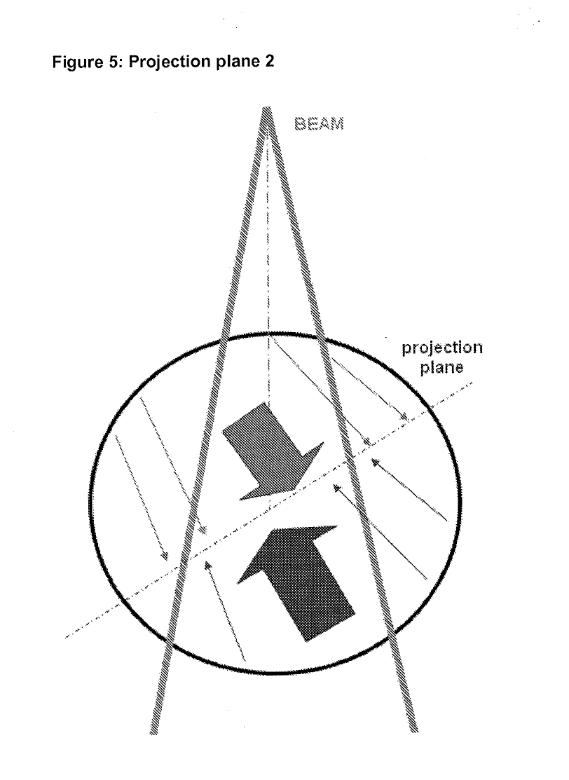

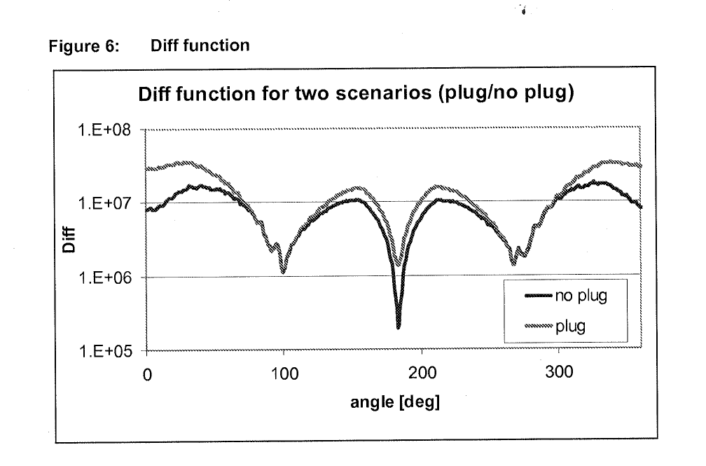

The projection plane transformation matrix contains angle and Y axis coordinate for each pixel location. It also contains attenuation factor for that angle and Y axis coordinate. The measurement can be done by open field measurement. FIG. 3 shows beam attenuation at Y=0 coordinate. The attenuation profile is in cylindrical coordinates. Each point represents one detector. Attenuation at certain angles between detectors can be interpolated from two neighboring detectors, only if such an interpolation follows the true attenuation profile. The similar profiles (similar to FIG. 3) have to be measured for all Y coordinates and stored in appropriate places in the projection plane transformation matrix.

Note: The FIG. 3 is presented in the cylindrical coordinates, which are α and β coordinates. The a coordinates correspond to angles 270-0-90 and to β coordinates correspond angles 90-270.

Using the Projection Plane Transformation Matrixes:

The goal is to calculate Diff function for all angles and locate the global minimum, which corresponds to the real source position. The Diff function (Equation 1) requires two images for each angle ω. The images are calculated using the plane transformation matrixes as:

image1_{i,j}(ω))=TM1(i,j,3)·MV(TM1(i,j,1),(TM1(i,j,2)+ω))

image2_{i,j}(ω)=TM2(i,j,3)·MV(TM2(i,j,1),(TM2(i,j,2)+ω)) Equation 10

ArcCHECK Coordinate System Not Aligned With the Linac Coordinate System

There are moments when the 3D detector is shifted or even rotated in some clinical cases. The ArcCHECK coordination system and Linac coordination system are no longer identical anymore and the above approach is modified.

Once the 3D detector is shifted, rotated or both, the system (Linac and 3D detector) is not invariant to rotation around Y axis anymore, unless the shift is done in the Y axis only. The shift in Y direction is not described here, because it is almost identical to the equations above.

Because the system is not invariant to rotation, the projection plane transformation matrix has 4 dimensions, instead of 3. The transformation matrix with shift and rotation can be presented as (TM_sr(K, L, 3, ω)), where K and L represent size of the projection plane. The third dimension contains three parameters: Shift of the point in Y axis, angle a and attenuation parameter (AT). The fourth dimension is angle of the gantry. The transformation matrix with shift and rotation (TM_sr) is like a TM matrix for each angle.

The TM_sr has to be calculated only once. It is advantageous to use the ArcCHECK coordination system for the TM_sr calculation. The method is the same as above, but with some modifications:

Taking the example of a 3D detector rotated around 3 different axis by angles τ, θ, φ and also shifted by a vector [Sx; Sy and Sz], the beam source coordinates in the Linac coordination system are:

$$Beam\_coordinates = T [ {x}_{2} ; {y}_{2} ; {z}_{2} ] = ( \begin{matrix}SAD \cdot sin ( ω ) \\ SAD \cdot cos ( ω ) \\ 0\end{matrix} ) .$$

To convert the Beam source coordinates into the ArcCHECK coordinate system:

$$\begin{matrix} ( \begin{matrix}{x}_{2}^{′} \\ {y}_{2}^{′} \\ {z}_{2}^{′}\end{matrix} ) = R ( τ , θ , φ ) \cdot ( \begin{matrix}SAD \cdot sin ( ω ) \\ SAD \cdot cos ( ω ) \\ 0\end{matrix} ) - ( \begin{matrix}Sx \\ Sy \\ Sz\end{matrix} ) , where & Equation 11 \\ R ( τ , θ , φ ) = ( \begin{matrix}cos ( φ ) & - sin ( φ ) & 0 \\ sin ( φ ) & cos ( φ ) & 0 \\ 0 & 0 & 1\end{matrix} ) \cdot ( \begin{matrix}cos ( θ ) & 0 & sin ( θ ) \\ 0 & 1 & 0 \\ - sin ( θ ) & 0 & cos ( θ )\end{matrix} ) \cdot ( \begin{matrix}cos ( τ ) & - sin ( τ ) & 0 \\ sin ( τ ) & cos ( τ ) & 0 \\ 0 & 0 & 1\end{matrix} ) & \end{matrix}$$

The projection plane is still located in the middle of ArcCHECK. Because the system is not invariant to rotation around Y axis, the projection plane has to rotate as well. The rotation should follow the gantry rotation, but it is not a requirement.

The following equation describes coordinates of the projection plane that is normal to the angle ω.

Projection plane coordinates: PP=PP[x₁;y₁;z₁]=PP[K·cos(ω);L;K·sin(ω)], Equation 12

ArcCHECK is described by Equation 2. Using beamlet equations (Equation 3) and transformed coordinates of the source, we can calculate:

((x′₂(ω)−x₁(ω))·t+x₁(ω))²+((z′₂(ω)−z₁(ω))·t+z₁(ω))²=radius² Equation 13

Solving for t:

t²·└(x′₂(ω)−x₁(ω))²+(z′₂(ω)−z₁(ω))²┘+t·2·[x₁(ω)·(x′₂(ω)−x₁(ω))+z₁(ω)·(z′₂(ω)−z₁(ω))]+x₁²(ω)+z₁²(ω)−radius²=0 Equation 14

Equation 14 results in two solutions for each beamlet at given angle.

Using results of the Equation 14 and Equation 3, one can calculate cross sections of the beamlet and ArcCHECK detector array. The result is in Cartesian coordinates and should be converted into cylindrical coordinates:

$$α ( ω ) = arc tan ( \frac{z}{x} ) = arc tan ( \frac{( {z}_{2}^{′} ( ω ) - {z}_{1} ( ω ) ) \cdot {t}_{1} + {z}_{1} ( ω )}{( {x}_{2}^{′} ( ω ) - {x}_{1} ( ω ) ) \cdot {t}_{1} + {x}_{1} ( ω )} ) \\ β ( ω ) = arc tan ( \frac{z}{x} ) = arctan ( \frac{( {z}_{2}^{′} ( ω ) - {z}_{1} ( ω ) ) \cdot {t}_{2} + {z}_{1} ( ω )}{( {x}_{2}^{′} ( ω ) - {x}_{1} ( ω ) ) \cdot {t}_{2} + {x}_{1} ( ω )} ) \\ {Y}_{1} ( ω ) = ( {y}_{2}^{′} ( ω ) - {y}_{1} ( ω ) ) \cdot {t}_{1} + {y}_{1} ( ω ) , and \\ {Y}_{2} = ( {y}_{2}^{′} ( ω ) - {y}_{1} ( ω ) ) \cdot {t}_{2} + {y}_{1} ( ω ) \qquad Equation 15$$

Attenuation Factor

Unless the shift or rotation is significant, the attenuation factors are very close to the default values. If greater accuracy is required, or if the shift and rotation are not negligible, the attenuation factors have to be measured or calculated for each specific system setup.

Using the Projection Plane Transformation Matrixes:

The goal is to calculate Diff function for all angles and locate the global minimum, which corresponds to the real source position. The Diff function (Equation 1) requires two images for each angle ω. The images are calculated using the projection plane transformation matrixes with shift and rotation as:

image1_{i,j}(ω)=TM1_{—}sr(i,j,3,ω)·MV(TM1_{—}sr(i,j,1,ω),TM1_{—}sr(i,j,2,ω))

image2_{i,j}(ω)=TM2_{—}sr(i,j,3,ω)·MV(TM2_{—}sr(i,j,1,ω),TM2_{—}sr(i,j,2,ω)) Equation 16

The foregoing description is provided for exemplary and illustrative purposes; the present invention is not necessarily limited thereto. Rather, those skilled in the art will appreciate that various modifications, as well as adaptations for particular circumstances, fall with the scope of the invention herein shown and described and of the claims appended hereto.

What is claimed is:

1. A method of radiation beam direction determination, the method comprising:
positioning a three-dimensional (3D) radiation measurement device in a radiation beam;
calculating direction of a primary radiation beam based on measured values from the 3D radiation measurement device and information about radiation attenuation in the 3D radiation measurement device.
